(19) United States
(12) Patent Application Publication
(10) Pub. No.: US 2007/0269399 A1
(43) Pub. Date: Nov. 22, 2007

(54) HYDROGEN PEROXIDE CONTAINING COMPOSITION

(75) Inventor: Sadao Tagawa, Aichi-ken (JP)

Correspondence Address: LAW OFFICE OF DELIO & PETERSON, LLC., 121 WHITNEY AVENUE, 3RD FLLOR, NEW HAVEN, CT, 06510, US

(73) Assignees: Sadao Tagawa, Aichi-ken (JP); Kazuo Tagawa, Osaka-hu (JP)

(21) Appl. No.: 11/682,374

(22) Filed: Mar. 6, 2007

(30) Foreign Application Priority Data
May 17, 2006 (JP) 2006-137879

(51) Int. Cl. A61K 8/28

(52) U.S. Cl. 424/74; 424/70.6

(57) ABSTRACT

A hydrogen peroxide containing composition, which sufficiently decreases the stimulation to the scalp, skin, and the like when used as a hair dye. The hydrogen peroxide containing composition includes allantoin, guaiazulene and aloe extract as the second agent for mixing with a first agent containing an oxidation dye. These ingredients in the hydrogen peroxide containing composition act synergistically and demonstrate an anti-inflammatory effect, an anti-allergic action, and a moisture retention effect.

### BACKGROUND OF THE INVENTION

1. Field of the Invention

This invention relates to a hydrogen peroxide containing composition applied to the hair.

2. Description of Related Art

Currently, a hydrogen peroxide containing composition is used as a medicine applied to the hair, such as a hair dye, hair bleaching and destaining agent, and permanent wave lotion.

Among these medicines, a two liquid mixing type oxidation type hair dye, which is used by mixing the first agent containing an oxidation dye and the second agent containing an oxidant just before hair dyeing, is widely utilized as a hair dye due to its fast color and many colors. The first agent of the general oxidation type hair dye (hereafter, called “the first agent”) is composed of an oxidation dye as a base compound and an alkaline chemical, and the like. The second agent of the oxidation hair dye (hereafter, called “the second agent”) is composed of an oxidant such as hydrogen peroxide and a small amount of stabilizer, and the like.

The alkaline chemicals in the first agent has the effect to swell and soften the hair, and also has the effect to decompose the oxidant in the second agent and to bleach the melanin pigment in the hair. The oxidant in the second agent has the effect to bleach the hair by decomposing the melanin pigment in the hair with the oxygen produced by the reaction with the alkaline chemicals in the first agent, and to colorize the hair by polymerizing the oxidation dye in the first agent and producing an insoluble pigment in the hair.

Therefore, it is essential that certain amounts of an alkaline chemical and oxidant are contained in the oxidation type hair dye in order to provide the function as a hair dye. In general, various kinds of alkaline chemicals having a pH from 8 to 11 are blended in the first agent, and the hydrogen peroxide as an oxidant from 3 to 9% is blended in the second agent.

However, for the scalp of the test subject, whose hair is treated with the above-mentioned hair dye, and the skin of the finger of the person applying the dye, which is used to confirm the dyeing condition, a reaction such as a stabbing pain may be felt or a redness may occur due to the alkaline chemical or oxidant included in the hair dye. In particular, a person who has a wound on his scalp, finger, and the like, and a person with sensitive skin may occasionally feel a strong reaction due to the alkaline chemical or the oxidant.

Currently, various measures have been proposed for hair dyes which decrease the stimulation to the scalp, and the like. Among these measures, for instance, in Japanese unexamined patent publication bulletin no. 2004-26699, a hair dye in which the stimulation to the scalp and the smell of ammonia are decreased by adjusting the pH during the treatment with monoethanolamine hydrochloride as a buffer included in the first agent, which includes ammonia as an alkaline chemical, is proposed.

Moreover, in Japanese unexamined patent publication bulletin no. 2002-363048, a hair dye in which the stimulation to the scalp is decreased by containing a water-soluble ammonium salt such as ammonium carbonate and polyvalent carbonic acid for controlling the pH in the first agent, which contains aqueous ammonia or mono-ethanolamine as the alkali chemical, is also proposed.

In the above-mentioned Japanese patent publications, the hair dye in which the stimulation to the scalp caused by the alkaline chemical is decreased by improving the first agent is proposed. However, in these references, the influence of the oxidant contained in the second agent has not been considered, and the stimulation to the scalp when used as a hair dye is not sufficiently decreased.

Moreover, currently, in most cases, the second agent was examined from the viewpoint of improvement in the stability and oxidizability, and was scarcely examined from the viewpoint of decreasing the stimulation to the scalp, and the like.

### SUMMARY OF THE INVENTION

Bearing in mind the problems and deficiencies of the prior art, it is an object of the present invention to provide a hydrogen peroxide containing composition, which sufficiently decreased the stimulation to the scalp, skin, and the like, when it is used as a hair dye.

As a result, it has been found that the stimulation to the scalp, skin, and the like, can be sufficiently decreased by utilizing allantoin, guaiazulene and the aloe extract in the hydrogen peroxide containing composition, which is used as the second agent. These ingredients act synergistically and demonstrate the anti-inflammatory effect, the anti-allergic action, and the moisture retention effect.

The above-mentioned object is achieved in the invention which is directed in one aspect to a first embodiment to a hydrogen peroxide containing composition applied to the hair, which contains hydrogen peroxide, allantoin, guaiazulene and the aloe extract.

In a second embodiment, the composition of the first embodiment may additionally contain at least one compound selected from the group consisting of tetradecene sodium sulfonate, 1-hydroxy ethane-1,1-diphosphonic acid or its salt, phosphoric acid, and disodium hydrogenphosphate.

In a third embodiment, the compositions of the aforementioned embodiments may additionally contain disodium edetate.

In a fourth embodiment, the compositions of the aforementioned embodiments may additionally contain a surface active agent and an oily ingredient.

In a fifth embodiment, the compositions of the aforementioned embodiments may additionally containing a synthetic color and/or a natural color.

The first embodiment of the hydrogen peroxide containing composition of this invention contains allantoin, guaiazulene and the aloe extract (hereafter, called the “three ingredients”) besides the hydrogen peroxide, and is used as the second agent of the hair dye. The stimulation and redness to the scalp, skin, and the like, can be decreased due to the anti-inflammation, antiallergic action, and the like, by the synergistic effect of these three ingredients. Therefore, based on this invention, the hydrogen peroxide containing composition, which can sufficiently decrease the stimulation to the scalp, skin, and the like, is provided.

In the second embodiment of this invention, it is desirable that the hydrogen peroxide containing composition contains at least one kind of compound selected from the group of tetradecene sodium sulfonate, 1-hydroxy ethane-1,1-diphosphonate or its salt, phosphoric acid, and disodium hydrogenphosphate, because the stability of the hydrogen peroxide can be improved by adjusting the pH of the hydrogen peroxide containing composition.

In the third embodiment of this invention, the hydrogen peroxide containing composition in which disodium edetate is additionally contained is desirable because the decomposition of hydrogen peroxide is prevented by masking the metal ion present in the hydrogen peroxide containing composition of this invention, and because the decrease in the oxidative power can be prevented.

In the fourth embodiment of this invention, the damage to the hair during hair dyeing can be reduced, and the gloss of hair can be retained when an oily ingredient is additionally present. Moreover, if a surface active agent is further included in addition to the oily ingredient, it is desirable because it stabilizes the oily ingredient by making an emulsion of the oily ingredient and also provides a uniform hair dyeing effect.

In the fifth embodiment of this invention, when the hydrogen peroxide containing composition also contains a synthetic color and/or natural color, it can be colored in order to easily distinguish the first, agent, therefore, this is desirable for determining the complete mixing condition during the mixing operation with the first agent and also for preventing any misuse with other medicines.

In another aspect, the present invention is directed to a hair dyeing composition comprising a mixture of a first agent containing an oxidation dye and a second agent containing any of the above-described embodiments of the hydrogen peroxide composition.

In a further aspect, the present invention is directed to a method of treating hair while decreasing stimulation to scalp or skin comprising providing a hair dyeing composition comprising a mixture of a first agent containing an oxidation dye and a second agent containing any of the above-described embodiments of the hydrogen peroxide composition, and contacting the hair with said hair dyeing composition.

### DESCRIPTION OF THE PREFERRED EMBODIMENTS

### Example 1

In the following Example 1, the hydrogen peroxide containing composition of this invention is applied as the second agent of the oxidation type hair dye is explained in detail. The second agent of this Example is the one used as an hair dye by mixing with the first agent, which contains the alkaline chemical, and contains hydrogen peroxide (A), allantoin, guaiazulene, and aloe extract (B). Hereinafter, the numeric value of the content is described as a weight percent of the total weight of the second agent unless otherwise noted.

Hydrogen peroxide (A) is an ingredient, which is blended as an oxidant in order to bleach the melanin pigment included in the hair and to oxidize the oxidation dye. The content of (A) is usually from about 0.2 to 15 weight percent, and desirably from about 2 to 9 weight percent, and more desirably from about 3 to 9 weight percent. When the content of (A) is less than 0.2 weight percent, the melanin pigment in the hair may not be sufficiently bleached, or the oxidation dye contained in the first agent may not be sufficiently oxidized. When the content of (A) exceeds 15 weight percent, some trouble may occur with the scalp, hair, and the like.

Component (B), which consists of allantoin, guaiazulene, and the aloe extract, possesses an anti-inflammation, antiallergic action, and the like, and can prevent the redness and the stimulation such as stabbing pains to the scalp, skin, and the like, by these effects.

In detail, allantoin has the effects to mainly suppress the inflammation and to relieve the itching. Giuaiazulene mainly has the anti-inflammatory effect, antiallergic action, and the like. The aloe extract mainly has a moisture retention effect, and the like. By blending these three ingredients (B) in the second agent, the anti-inflammatory effect and the antiallergic action can be synergistically exhibited, and not only the stimulation caused by the second agent, but also the stimulation attributed to the first ingredient are decreased.

The desirable content of allantoin is from about 0.1 to 0.3 weight percent, and more desirably from about 0.1 to 0.2 weight percent. The desirable content of guaiazulene is from about 0.01 to 0.05 weight percent, and more desirably from about 0.01 to 0.02 weight percent. The desirable content of the aloe extract is from about 0.01 to 0.2 weight percent, and more desirably from about 0.1 to 0.2 weight percent.

From the viewpoint that the function of the second agent of this Example is to improve the stability of component (A) by adjusting the pH, at least one kind of component (C) selected from the group of tetradecene sodium sulfonate, 1-hydroxy ethane-1,1-diphosphonic acid, its salt, phosphoric acid, and disodium hydrogenphosphate is desirably contained in the second agent.

The desirable content of component (C) is from about 0.002 to 1 weight percent, and more desirably from about 0.005 to 0.2 weight percent. When the content of the component (C) is less than 0.002 weight percent, the pH control effect may not be sufficiently obtained. On the other hand, when the content of the component exceeds 1 weight percent, the pH control effect cannot be further improved.

The pH of the second agent in this Example is desirably acidic, and more desirably in the range of pH 2 to 6 from the viewpoint of stability of component (A). On the other hand, when the pH is alkaline, the stability of component (A) might be degraded.

As for the second agent of this Example, it is desirable that component (D) mask the activity of the metal ions contained in it. The reason for this is that the deterioration of component (A) starts due to the initiation of an activation reaction by the metal ions, and that the oxidative power gradually decreases and the efficacy of the second agent decreases, and that it is dangerous because the oxygen gas generated by the decomposition of component (A) is accumulated in the vessel which increases the internal pressure of the vessel.

As a specific example of component (D), disodium edetate is presented. The content of component (D) is usually from about 0.01 to 0.05 weight percent, and desirably from about 0.01 to 0.02 weight percent.

As for the solvent of the second agent in this Example, water (E) is used for dissolving the above mentioned components (A) through (D) to make an aqueous solution, being used as a dispersant or emulsifier, and used for making the pharmaceutical preparation. The content of (E) is desirably from about 50 to 98 weight percent, and more desirably from about 75 to 95 weight percent. When the content of (E) is less than 50 weight percent, the formation of the aqueous solution, dispersant or emulsifier may not be stably obtained, and when it exceeds 98 weight percent, it may become difficult to maintain the homogeneity of the second agent and to carry out the pharmaceutical preparation.

As for the second agent of this Example, it is desirable that an oily ingredient is contained in it from the viewpoints of protection against the damage to the hair during the hair dyeing and of gloss retention. Moreover, it is more desirable that a surface active agent is additionally contained from the viewpoints of stabilizing the oily ingredient by making it an emulsion and of providing a uniform hair dyeing effect.

Polyhydric alcohol, higher alcohol, hydrocarbon, waxes, oils and fats, higher fatty acids, their esters, silicons, and the like, are enumerated as examples of the oily ingredient. In the following section, the one suitably usable in this invention is described.

The glycols such as ethylene glycol, propylene glycol, butylene glycol, and the glycerins such as glycerin, diglycerol, are enumerated as examples of the polyhydric alcohol. Lauryl alcohol, cetyl alcohol, stearyl alcohol, lanolin alcohol, and the like, are enumerated as examples of the higher alcohol. Liquid paraffin, synthetic squalane, vaseline, squalane, paraffin, and the like, are enumerated as examples of the hydrocarbon. Propolis, carnauba wax, lanolin and the like, are enumerated as examples of the waxes. Castor oil, soybean oil, beef tallow, oil of almonds, yolk oil, mink oil, and the like, are enumerated as examples of the oils and fats. Lauric acid, palmitic acid, stearic acid, oleic acid, isostearic acid, lanolin fatty acid, and the like, are enumerated as examples of the higher fatty acid. Isopropyl myristate, isopropyl palmitate, butyl stearate, lanolin derivative, and the like, are enumerated as examples of the esters of the higher fatty acid. Dimethyl polysiloxane, amino modified silicone, polyether modified silicone, and the like, are enumerated as examples of the silicones.

Though these oily ingredients can be independently used or as a mixture of multiple ingredients, it is preferable to use the mixture of several ingredients. In particular, it is more desirable to use at least one kind of polyhydric alcohol in order to achieve a moist efficacy, homogeneity, and stability of the second agent.

The content of the oily ingredient is desirably from about 0.1 to 15 weight percent, and more desirably from about 1 to 10 weight percent. When the content is less than 0.1 weight percent, there is a possibility that the stability of the emulsification and the feeling of moisture and flexibility of the hair is not obtained. On the other hand, when the content exceeds 15 weight percent, the hair dyeing effect may not be sufficiently obtained.

A nonionic surface active agent, cationic surface active agent, anionic surface-active agent, and amphoteric surface active agent are used as the surface active agent. In the following section, the one, which can be suitably used in this invention, is exemplified

Polyoxyethylene (hereafter, called “POE”), lauryl ether, the POE alkylethers, such as POE cetyl ether, POE stearyl ether, and the like, the POE alkyl phenol ethers, the POE sorbitan fatty acid esters and the POE propylene glycol fatty acid esters are enumerated as examples of the nonionic surface active agent.

Lauryl trimethylammonium chloride, stearyl trimethylammonium chloride, and the like, are enumerated as examples of the cationic surface active agent.

The alkyl sulfates such as sodium lauryl sulfate, the POE alkyl sulfates such as sodium POE lauryl ether sulfate, and the like, are enumerated as examples of the anionic surface-active agent.

Lauryl dimethylamino betaine acetate, cocoamidopropyl betaine, and the like, are enumerated as examples of the amphoteric surface active agent. These surface active agents can be used singly or together.

The content of the surface active agent is desirably from about 0.1 to 10 weight percent, and more desirably from about 1 to 5 weight percent. When the content is less than 0.1 weight percent, the stability of the second agent might become insufficient. On the other hand, when the content exceeds 10 weight percent, the hair dyeing force or strength might decrease.

It is desirable that a direct dye such as the synthetic color, natural color, and the like, is included in the second agent of this Example. The reason for this is as follows.

The conventional second agent has a shortcoming that the complete mixing condition was hardly determined when the first agent and the second agent were mixed because most of the second agent was a transparent and colorless liquid or white cream. Moreover, many kinds of the second agent with different concentrations of oxidant are being sold, and they are used by taking the property of the subject and the coloring condition into consideration. However, the difference in the color tone due to the difference in the concentration is not clear, and there was the possibility to be misused as another agent with a different concentration.

Because the second agent of this Example has a milk-white color with an ice-blue tint derived from guaiazulene when it does not contain the direct dye, it is easy to determine the condition of the complete mixing with the first agent when compared with the conventional second agent. However, the determination of the complete mixing condition with the first agent can be carried out more easily by adding the direct dye to the second agent of this Example. In addition, because another second agent with a different color tone can be provided depending on the concentration of the hydrogen peroxide of the second agent by changing the kind and amount of the direct dye contained in the second agent, the misuse with other chemicals can be prevented.

The synthetic color, natural color, and the like, are used as the direct dye, and they can be used as a single ingredient or in combination. As for the synthetic color, the one that is registered as a synthetic color usable for medicine, and the like, (The Japanese Ministry of Health, Labor and Welfare notification No. 126 in 2003), can be used. As for the natural color, cochineal, and the like, can be used. The direct dye is usually contained from about 0.001 to 0.01 weight percent.

Ingredients other than the above-mentioned ones, for instance, a stabilizer such as peacetime, water-soluble polymer compound, viscosity adjustment chemicals, moisturizing agent, antistatic agent, hair softening agent, oxidation inhibitor, diluting agent, perfume, and the like, can be contained in the second agent of this Example. As for the first agent, which is mixed with the second agent of this Example, various conventional agents can be used without any limitations

### Examples 1 Through 6, Comparison Examples 1 Through 3

Next, the above-mentioned Example 1 is more specifically explained by describing a practical example and the comparison example.

(1) Preparation of the First Agent

The first agent of the two-liquid mixing type hair dye was prepared by the composition indicated in Table 1. The numbers in the table indicate the weight percent to the entire weight of the first agent.

TABLE 1

| Ingredient | Weight % |
|---|---|
| 28% aqueous ammonia | 5 |
| Monoethanolamine | 1 |
| POE cetyl ether | 8 |
| Cetyl alcohol | 10.5 |
| Stearyl trimethyl ammonium chloride | 0.5 |
| Ammonium hydrogencarbonate | 0.8 |
| Paraphenylendiamin | 1 |
| Resorcin | 0.8 |
| Para-aminophenol | 0.2 |
| Ammonium thioglycolate | 0.1 |
| Propylene glycol | 3 |
| Water | residue |

(2) Preparation of the Second Agent

The second agents of Examples 1 through 6 (this invention) and Comparison Examples 1 through 3 (for comparison) were prepared according to the composition listed in Table 2. In addition, the second agent of this invention and the comparison were prepared using the blue dye No. 404 as the synthetic color in order to be easily distinguished from the first agent. The numbers in the table indicate the weight percent to the entire weight of the second agent.

TABLE 2

| | | | | | | | Comparison | | |
|---|---|---|---|---|---|---|---|---|---|
| | Example | | | | | | example | | |
| | 1 | 2 | 3 | 4 | 5 | 6 | 1 | 2 | 3 |
| Ingredient | | | | | | | | | |
| A, E) 35% Hydrogen | 17 | 17 | 17 | 17 | 17 | 17 | 17 | 17 | 17 |
| peroxide | | | | | | | | | |
| B) Guaiazulene | 0.02 | 0.02 | 0.01 | 0.02 | 0.01 | 0.02 | 0 | 0 | 0 |
| B) Allantoin | 0.2 | 0.2 | 0.2 | 0.1 | 0.2 | 0.1 | 0 | 0 | 0 |
| B) Aloe extract | 0.2 | 0.1 | 0.1 | 0.2 | 0.2 | 0.1 | 0 | 0 | 0 |
| C) Tetradecene sodium | 0.05 | 0.05 | 0.1 | 0.1 | 0.1 | 0.1 | 0.05 | 0 | 0.1 |
| sulfonate | | | | | | | | | |
| C) 1-hydroxy ethane-1,1- | 0 | 0.1 | 0 | 0.1 | 0 | 0.1 | 0.1 | 0.1 | 0.1 |
| disulfonate | | | | | | | | | |
| C) Phosphoric acid | 0.05 | 0 | 0.05 | 0 | 0.05 | 0.05 | 0.05 | 0.05 | 0.05 |
| C) Disodium hydrogen | 0.05 | 0 | 0.05 | 0 | 0.05 | 0.05 | 0 | 0.05 | 0 |
| phosphate | | | | | | | | | |
| D) Disodium edetate | 0.01 | 0.01 | 0.02 | 0.02 | 0.01 | 0.01 | 0.01 | 0.02 | 0.01 |
| Cetyl alcohol | 2 | 2 | 2 | 2 | 2 | 2 | 2 | 2 | 2 |
| Stearyl trimethyl | 0.2 | 0.2 | 0.2 | 0.2 | 0.2 | 0.2 | 0.2 | 0.2 | 0.2 |
| ammonium chloride | | | | | | | | | |
| Liquid paraffin | 0.8 | 0.8 | 0.8 | 0.8 | 0.8 | 0.8 | 0.8 | 0.8 | 0.8 |
| POE cetyl ether | 0.5 | 0.5 | 0.5 | 0.5 | 0.5 | 0.5 | 0.5 | 0.5 | 0.5 |
| Propylene glycol | 1 | 1 | 1 | 1 | 1 | 1 | 1 | 1 | 1 |
| POE sodium lauryl sulfate | 0.2 | 0.2 | 0.2 | 0.2 | 0.2 | 0.2 | 0.2 | 0.2 | 0.2 |
| Squalane | 0.1 | 0.1 | 0.1 | 0.1 | 0.1 | 0.1 | 0.1 | 0.1 | 0.1 |
| Phenacetin | 0.1 | 0.1 | 0.1 | 0.1 | 0.1 | 0.1 | 0 | 0.1 | 0 |
| Synthetic color | trace | trace | trace | trace | trace | trace | trace | trace | trace |
| E) Purified water | remainder | | | | | | remainder | | |
| Evaluation | | | | | | | | | |
| Stimulation to the skin | a | a | b | a | b | b | d | d | d |
| Stimulation to the scalp | a | a | b | a | b | b | d | d | d |
| Redness on the skin | a | a | a | a | a | a | a | c | d |

Example of Testing

The hair dyes were prepared by mixing the first agent with the second agent of Examples 1 through 6 and Comparison Examples 1 through 3 at a weight ratio of 1:1, and they were tested as follows.

(1) Scalp Hair Dyeing Test

Thirty-five adults with some gray hair were selected as the test subjects. The hair dyeing tests were carried out under the same conditions of hair dyeing treatment such as the hair dyeing time, the temperature, and the like, for each case when Examples 1 through 6 or Comparison Examples 1 through 3 was used as the second agent to the same test subject.

(a) Evaluation of Stimulation to the Scalp and Skin

The hair dyeing treatments using the second agent of this invention and the one using the comparative second agent were carried out at an interval of more than two months. The stimulation to the scalp that the test subject felt and the stimulation to the skin that the applier felt during each hair dyeing treatment were evaluated.

The evaluation standard of the stimulation for each second agent used in this test was set as follows. Grade 3: no stimulation. Grade 2: close to no stimulation. Grade 1: slight stimulation. Grade 0: considerable stimulation.

The average value was calculated by dividing the total of the grade points by the number of subjects. The results are shown in Table 2, where “a” denotes the average value of over 2.3, “b” denotes 1.8 to 2.3, “c” denotes 1.3 to 1.8, and “d” denotes less than 1.8.

(b) Observation of Redness on the Scalp

In addition to the evaluation of the stimulation to the scalp and skin, the redness on the scalp of the subjects was observed. The rate of the subjects who had a redness to the entire number of subjects was calculated for each second agent used in this test. The results are shown in Table 2, where, “a” is 0%, “b” is less than about 10%, “c” is from about 10% to 15%, and “d” is over about 15%.

Results and Consideration

(1) As shown in Table 2, excellent results with a slight stimulation and no redness on the scalp of the subject and finger of the applier were obtained for the second agent of this invention different from the Comparative Example.

The reason for this is considered that the anti-inflammatory effect and the antiallergic action were demonstrated by the effect of component (B), which included allantoin, guaiazulene, and the aloe extract, in this invention.

(2) In particular, excellent results were obtained for Examples 1, 2, and 4 of this invention. In Examples 1, 2, and 4, it is understood that the effect to decrease the stimulation was influenced by the guaiazulene content because they have higher amount of guaiazulene compared to Examples 3 and 5.

However, as for Example 6, the effect to decrease the stimulation is inferior to Examples 1, 2, and 4 though the guaiazulene content is equal to that of Examples 1, 2, and 4. The reason for this can be presumed that the content of allantoin and the aloe extract in Example 6 is lower than that in Examples 1, 2, and 4. Based on these results, it can be presumed that the best effect in this invention was achieved by the synergistic action of the three ingredients of guaiazulene, allantoin, and the aloe extract.

Therefore, a hydrogen peroxide containing composition, which sufficiently decreases the stimulation to the scalp and skin, can be provided according to this invention.

### Other Examples

The scope of this invention is not limited to the Examples explained by the above-mentioned description and drawings, but for instance, the following Examples are included within the technical scope of this invention.

(1) The second agent in the above-mentioned Examples 1 through 6 was the one, which contained the oily ingredient and surface active agent listed in Table 2. However, a second agent, which contains another oily ingredient and surface active agent is also usable.

(2) Though the second agent of Examples 1 through 6 contained the synthetic color, the one, which contains the natural color or another kind of synthetic color, is also usable.

(3) Though the concentrations of hydrogen peroxide in the second agent of Examples 1 through 6 were the same, the one, which has a low concentration, is usable. Furthermore, the color tone of the second agent may be changed by adjusting the amount or kind of the direct dye in it depending on the difference in the concentration of hydrogen peroxide.

While the present invention has been particularly described, in conjunction with specific preferred embodiments, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A hydrogen peroxide containing composition which is applied to the hair, comprising hydrogen peroxide, allantoin, guaiazulene, and aloe extract.

2. The hydrogen peroxide containing composition of claim 1 further including at least one compound selected from the group consisting of sodium tetradecene sulfonate, 1-hydroxy ethane-1,1-diphosphonic acid or its salt, phosphoric acid, and disodium hydrogenphosphate.

3. The hydrogen peroxide containing composition of claim 1 further including disodium edetate.

4. The hydrogen peroxide containing composition of claim 2 further including disodium edetate.

5. The hydrogen peroxide containing composition of claim 1 further including a surface active agent and an oily ingredient.

6. The hydrogen peroxide containing composition of claim 2 further including a surface active agent and an oily ingredient.

7. The hydrogen peroxide containing composition of claim 3 further including a surface active agent and an oily ingredient.

8. The hydrogen peroxide containing composition of claim 1 further including a synthetic color and/or a natural color.

9. The hydrogen peroxide containing composition of claim 2 further including a synthetic color and/or a natural color.

10. The hydrogen peroxide containing composition of claim 3 further including a synthetic color and/or a natural color.

11. The hydrogen peroxide containing composition of claim 5 further including a synthetic color and/or a natural color.

12. The hydrogen peroxide containing composition of claim 1 wherein the allantoin is present in an amount of about 0.1 to 0.3 weight percent of the composition, the guaiazulene is present in an amount of about 0.01 to 0.05 weight percent of the composition, and the aloe extract is present in an amount of about 0.01 to 0.2 weight percent of the composition.

13. The hydrogen peroxide containing composition of claim 1 wherein the allantoin is present in an amount of about 0.1 to 0.2 weight percent of the composition, the guaiazulene is present in an amount of about 0.01 to 0.02 weight percent of the composition, and the aloe extract is present in an amount of about 0.1 to 0.2 weight percent of the composition.

14. A hair dyeing composition comprising a mixture of a first agent containing an oxidation dye and a second agent containing hydrogen peroxide, allantoin, guaiazulene, and aloe extract.

15. The composition of claim 14 wherein the second agent further includes at least one compound selected from the group consisting of sodium tetradecene sulfonate, 1-hydroxy ethane-1,1-diphosphonic acid or its salt, phosphoric acid, and disodium hydrogenphosphate.

16. The composition of claim 14 wherein the second agent further includes disodium edetate.

17. The composition of claim 14 wherein the second agent further includes a surface active agent and an oily ingredient.

18. The composition of claim 14 wherein the second agent further includes a synthetic color and/or a natural color.

19. The composition of claim 15 wherein the second agent further includes a synthetic color and/or a natural color.

20. A method of treating hair while decreasing stimulation to scalp or skin comprising:
providing a hair dyeing composition comprising a mixture of a first agent containing an oxidation dye and a second agent containing hydrogen peroxide, allantoin, guaiazulene, and aloe extract; and
contacting the hair with said hair dyeing composition.
